(19) United States
(12) Patent Application Publication
(10) Pub. No.: US 2011/0008909 A1
(43) Pub. Date: Jan. 13, 2011

(54) Assay Device

(76) Inventors: James Homrig, San Diego, MA (US); Robert Klepper, San Diego, CA (US); David Smart, Moira (GB)

Correspondence Address: FOLEY HOAG, LLP (w/ISA);PATENT GROUP, 155 SEAPORT BLVD., BOSTON, MA, 02210-2600, US

(21) Appl. No.: 12/681,545

(22) PCT Filed: Oct. 3, 2008
(86) PCT No.: PCT/GB2008/003368

§ 371 Date: Jul. 19, 2010

(30) Foreign Application Priority Data
Oct. 3, 2007 (GB) 0719375.8

(51) Int. Cl. G01N 33/566; C12M 1/34; G01N 30/62

(52) U.S. Cl. 436/501; 435/287.1; 422/69

(57) ABSTRACT

Disclosed is an assay device for the determination of the presence and/or amount of a species in a liquid sample, the assay device comprising one or more reagents for providing an analyte of interest wherein the assay device further comprises a control species which is capable of interacting with the one or more reagents to provide a detectable control antigen.

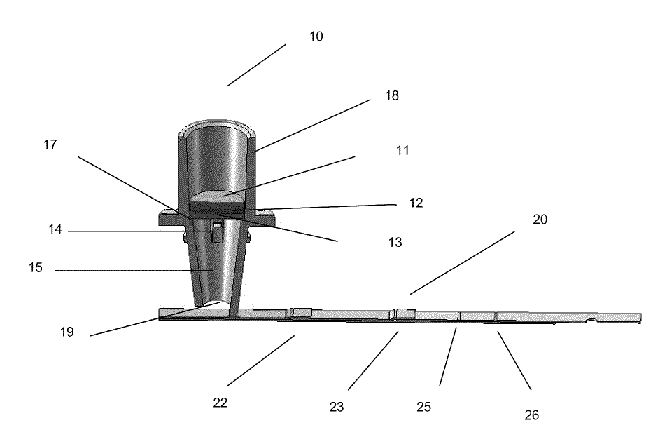

The present invention relates to an assay device, method and kit for detecting the presence and/or amount of an analyte in liquid sample. In particular it relates to an assay device, method or kit comprising an assay control.

Simple lateral flow immunoassay devices have been developed and commercialized for detection of analytes in liquid samples, see for example EP291194. Such devices typically comprise a porous carrier comprising a dried mobilisable labelled binding reagent capable of binding to the analyte in question, and an immobilised binding reagent also capable of binding to the analyte provided at a detection zone downstream from the labelled binding reagent. Detection of the immobilised labelled binding reagent at the detection zone provides an indication of the presence of analyte in the sample. Devices such as the above device are suitable for the detection of analytes, such as hCG or drugs of abuse, in urine. The lateral flow device disclosed by EP291194 may also comprise a control zone to indicate whether the test has been carried out correctly. The control zone may for example comprise an immobilised binding reagent which is capable of binding a labelled binding reagent in either the presence or absence of the analyte of interest.

However, detection of some analytes by a simple immunoassay requires a pre-treatment step in order to extract the analyte, in particular in the detection of pathogenic organisms which have a protective coating enclosing the analyte of interest. For example, the detection of organisms, such as from the family Streptococcae, typically relies on the detection of a specific carbohydrate antigen. The antigenic molecules must first be released from the cell wall of the organism before they can be detected. A number of methods exist for carrying out such extraction treatments using, for example, nitrous acid, ProNase B enzyme, hot formamide or hot HCL. The majority of immunoassay tests that utilise lateral flow technology, for example, utilise nitrous acid for the extraction of the carbohydrate antigen from the organism before contacting the sample containing the extracted antigen with the assay device. Nitrous acid, however, is chemically unstable and needs to be generated.

EP231750 discloses a test kit for extracting a carbohydrate antigen from a swab comprising a test sample of suspected of containing Streptococcus A. The test kit comprises a first vessel containing a polymeric acid and a second vessel comprising sodium nitrite solution. The sodium nitrite solution is added to the vessel containing the polymeric acid followed by addition of the test sample in order to extract the carbohydrate antigen. The extraction of carbohydrate antigen and its subsequent detection would generally be as follows: a swab sample is taken from the back of the throat for example and the swab inserted into a vial containing nitrous acid generated by the addition of aqueous solution of sodium nitrite and acetic acid. The swab may subsequently agitated to release the antigens into solution.

WO2006/013329 discloses a lateral flow assay device for determining the presence of Streptococcus Group A antigen in a sample comprising a dried polysulphonic acid reagent and dried sodium nitrite provided within the porous carrier. Addition of liquid sample to the assay test-strip results in the in situ generation of nitrous acid. The assay strip may further comprise an assay control similar to that disclosed by EP291194.

Species-specific analytes or antigens of many pathogenic organisms other than from the family Streptococcae require pre-treatment prior to detection. For example, in the detection of influenza A or B, the virus requires pre-treatment with an extraction agent in order to disrupt the viral particles and expose the internal viral nucleoproteins. Legionella pneumophila may be detected by non-serotype specific monoclonal antibodies after pre-treatment with detergents and EDTA. Pseudomonas aeruginosa also requires treatment by detergents and EDTA in order to optimally detect Porin F protein antigens. Microbial pathogens may be found in seeds, fruit and vegetables. Increased sensitivity has been reported using a lysozyme extraction buffer in the detection of Xanthomona campestri pv vescatoria in plant tissue by immunoassay (J. B. Jones, G. C. Somodi and J. W. Scott; Journal of Applied Microbiology 1997, 83, 397-401). The analyte of interest may be DNA, which may be extracted from E. coli O157:H7, an important food-borne pathogen, and detected by PCR. U.S. Pat. No. 5,731,162 discloses an immunoassay for the detection of at least two micro-organisms selected from the group consisting of Chlamydia trachomatis, Neisseria gonorrhoea and Mycoplasma comprising incubating a biological sample in an extraction reagent consisting of proteinase K and a lipase.

However, the assay controls disclosed thus far for use with assay devices or kits employing a reagent pre-treatment step are limited in their ability to determine whether the assay has been carried out correctly, in that the assay control is not able to determine whether the pre-treatment step has actually been carried out or whether it has been carried out effectively.

In certain embodiments, the invention provides an assay device, kit and method comprising an improved assay control.

According to a first aspect, the invention provides a method for determining the presence and/or amount of an analyte of interest in a liquid sample, comprising:
(a) applying the sample to an assay device comprising one or more reagents and a control substance which comprises a control analyte, wherein the one or more reagents interacts with the liquid sample and with the control substance to provide the analyte of interest and the control analyte in a detectable or more detectable form;
(b) causing the analyte of interest to interact with a first labelled binding reagent and the control analyte to interact with a second labelled binding reagent;
(c) detecting the first labelled binding reagent at a detection zone and the second labelled binding reagent at a control zone;
wherein detection of the first labelled binding reagent at the detection zone is indicative of the presence or amount of the analyte of interest and wherein detection of the second labelled binding reagent at the control zone is indicative of the effectiveness of the one or more reagents in providing the analyte of interest in a detectable or more detectable form.

Thus, in certain methods of the invention, liquid sample applied to the assay device interacts with the one or more reagents to provide the analyte of interest. The same reagent(s) also provide(s) the control analyte. The treated liquid sample thereafter interacts with the labelled binding reagents which are conveyed, preferably downstream, along with the liquid sample to the detection and control zones. Detection of the labelled binding reagents at the detection and control zones is respectively indicative of the amount and/or presence of the analyte of interest and the control analyte. The liquid sample may undergo a period of incubation prior to contacting the labelled reagents. The liquid sample may also undergo a buffer treatment by interaction with a buffer reagent prior to contacting the labelled binding reagents in order to bring the pH of the sample liquid to within an optimal pH range of the immunoassay.

According to a second aspect, the invention provides an assay device for determining the presence and/or amount of an analyte of interest in a liquid sample, said assay device comprising a liquid flow-path comprising:
(a) one or more reagents which, when contacted with the liquid sample, provide the analyte of interest in a detectable or more detectable form;
(b) a control substance, comprising a control analyte, which, when contacted with said one or more reagents provides the control analyte in a detectable or more detectable form;
(c) a detection zone for detecting a first labelled binding reagent for the analyte of interest, provided downstream from the one or more reagents; and
(d) a control zone for detecting a second labelled binding reagent for the control analyte, provided downstream from the control analyte and from the one or more reagents;
wherein detection of the first labelled binding reagent at the detection zone is indicative of the presence and/or amount of the analyte of interest and detection of the second labelled binding reagent at the control zone is indicative of the effectiveness of the one or more reagents in providing the analyte of interest in a detectable or more detectable form.

According to a third aspect, the invention provides an assay kit for the determination of the presence and/or amount of an analyte of interest in a liquid sample, comprising:
(a) a control substance comprising a control analyte;
(b) one or more reagents for providing the analyte of interest and the control analyte in a detectable or more detectable form;
(c) a first labelled binding reagent for the analyte of interest and a second binding reagent for the control analyte; and
(d) an assay device for receiving the liquid sample comprising a liquid flow-path comprising a detection zone for detecting the first labelled binding reagent and a control zone for detecting the second labelled binding reagent.

The assay device of the present invention comprises one or more liquid flow-paths along which liquid may flow, such as a channel, a porous carrier such as a vertical flow or lateral flow porous carrier, or a combination, e.g. of a channel and a porous carrier. The assay flow-path may further comprise other fluidic elements such as a liquid chamber, a valve, a filter and a time gate.

The porous carrier may comprise one or a plurality of porous carrier materials which may overlap in a linear or stacked arrangement or which are otherwise fluidically connected. The porous carrier materials may be the same or different. The porous carrier may comprise the detection zone and the control zone. In addition, the one or more reagents and/or the control analytes may be provided on and/or in the porous carrier.

In one embodiment, the analyte of interest is associated with a species of interest. The one or more reagents may serve to interact or react with the species of interest in order to provide an analyte of interest in a detectable or more detectable form. For example, the analyte of interest may reside within the protective coating or cell wall of an organism, thus making it unavailable or less available to bind to a binding reagent. The one or more reagents may serve to extract an analyte of interest. Extraction refers to the processing of a sample so as to make a marker more accessible for measurement. By way of example, extraction includes the liberation and/or solubilisation of markers from cells, micro-organisms or organelles, e.g., by (i) rupturing or solubilising membranes, cell walls, envelopes, etc. to release markers comprised or encased within, attached to and/or incorporated into the membranes, cell walls, envelopes, etc., (ii) cleaving a marker from a larger chemical moiety, (iii) breaking down and/or dissolving a polysaccharide coat and/or (iv) breaking down and/or dissolving a jelly coat. Extraction also includes the liberation of markers, cells, organelles and/or micro-organisms from components of the surrounding sample matrix. The matrix may include the medium in which the organism or the marker is present.

Alternatively, the analyte of interest may already be present in the liquid sample in a detectable form, the one or more reagents serving to provide it in a more detectable form, by for example increasing its solubility or by changing the operating pH of the assay, by an enrichment step or by treatment with an enzyme. A plurality of reagents may be provided as reagent precursors, which, when combined, form the reagent of interest. The analyte of interest provided in a detectable or more detectable form may be the same as the analyte prior to treatment with the one or more reagents or it may be an analyte analogue, formed as a consequence of the reagent treatment.

The one or more reagents may be provided in the dry state.

The one or more reagents may comprise by way of example an acid, a buffer, a detergent, an enzyme, reagent precursors such as an acid and an alkali metal nitrite for the generation of nitrous acid, a resin, a fibrin lysis reagent.

The control substance may comprise or consist of the control analyte, When treated with the one or more reagents, the control substance provides the control analyte in a detectable or more detectable form. Alternatively, the control substance may comprise or consist of a control analyte precursor, which when treated with the one or more reagents, provides the control analyte in a detectable form. In certain embodiments of the invention, the control substance is a control species, i.e. the control analyte is present in or associated with the control species. The control species is one which may be treated with the one or more reagents in order to provide a detectable or more detectable control analyte. The one or more reagents may serve to extract the control analyte from the control species. This extraction may be in the same manner as the analyte of interest is extracted. Alternatively, the control analyte may be provided in a more detectable form by the one or more reagents in the same manner as the analyte of interest. Thus, detection of the control analyte at the control zone indicates that the reagent pre-treatment has worked effectively and is thus indicative that the reagent pre-treatment for the provision of the analyte in a detectable or more detectable form has also been effective. An advantage of such a control zone is that it is able to indicate the effectiveness of the one or more reagents in providing a detectable or more detectable analyte of interest (or analogue thereof). The control zone is additionally able to indicate that liquid sample has been applied to the device, that the labelled binding reagent has been resuspended and conveyed to the control zone and that the control zone is capable of detecting said labelled binding reagent. Thus, where an analyte pre-treatment step is required, the control zone is able to provide a further level of confidence that the assay has been carried out correctly. The level of the control analyte or control species may be chosen such that it corresponds to a lower limit of the assay range for the analyte of interest. An advantage of this is that detection of control analyte at the control zone is indicative that the one or more reagents are working sufficiently well at low levels of analyte. The control analyte may also be provided as part of the assay flow path and provided upstream from the detection and control zones. The control analyte may be provided upstream, downstream from the one or more reagents or in the vicinity of them. Alternatively or additionally, the signal at the control zone may be used to calibrate the signal at the detection zone.

Detection of the analyte of interest and the control analyte at the detection and control zones respectively may comprise immobilisation of the respective first and second labelled binding reagents. Thus, the detection zone may comprise an immobilised binding reagent for immobilising the labelled binding reagent for the analyte of interest or a labelled binding reagent-analyte of interest complex. The control zone may comprise an immobilised binding reagent for immobilising the labelled binding reagent for the control analyte or a labelled binding reagent-control analyte complex. The control zone may be advantageously provided downstream from the detection zone. Thus detection of labelled binding reagent at the control zone is indicative that it has been conveyed beyond the detection zone.

As an alternative to providing immobilised binding reagent at the detection zone, the binding reagent may be provided in a mobilisable form which is capable of binding to an analyte of interest-labelled binding reagent complex. The binding reagent may for example be conjugated to a large particle, such as agarose, and the detection zone may comprise a filter whose pore-size has dimensions smaller than the large particle, but larger than the size of the labelled binding reagent, such that the filter is able to trap the any labelled binding reagent-analyte-binding reagent complex present, with any labelled binding reagent that is not complexed to the capture reagent being able to pass through the filter. Yet alternatively a reagent may be provided in an immobilised form at the detection zone that is capable of binding a mobilisable labelled binding reagent-analyte-binding reagent complex. For example the binding reagent may be provided in a mobilisable form and conjugated to a binding species such as biotin, the reagent immobilised at the detection zone being a complementary binding partner such as streptavidin. The considerations described above in respect of the detection zone may also apply to the control zone.

The assay device of the invention may comprise labelled binding reagent for the analyte of interest and/or labelled binding reagent for the control analyte. Where labelled binding reagents for the analyte of interest and for the control analyte are present as part of the assay device, they are provided downstream from the one or more reagents and upstream respectively from the detection and control zones. Both labelled reagents may be provided upstream from the detection and control zones. The labelled binding reagent for the control analyte is advantageously one which is not detectable at, e.g. does not bind or does not substantially bind to, the detection zone. Likewise the labelled binding reagent for the control analyte is advantageously one which does not bind or substantially bind to the analyte. The labelled binding reagents may be provided downstream from the control analyte and from the one or more reagents.

In one embodiment, a first porous carrier material comprises the first and second labelled binding reagents provided upstream from a second porous carrier material comprising the detection and control zones. A buffer zone may be provided downstream from the one or more reagents and upstream from the first and second labelled binding reagents. The buffer zone may be provided on a separate porous carrier material to and upstream from the first and second porous carrier materials.

The assay may be of the sandwich type, namely the labelled binding reagent binds to analyte to form a labelled binding reagent-analyte complex which is subsequently immobilised at the detection zone by an immobilised binding reagent for the analyte. Alternatively, in particular when the analyte of interest is a hapten, a labelled analyte or labelled analyte analogue may be provided in a mobilisable form upstream from an immobilised binding reagent at a detection zone. Yet alternatively, the assay device may employ an inhibition reaction wherein an immobilised analyte or analyte analogue is provided at the detection zone, the assay device further comprising a mobilisable labelled binding reagent for the analyte provided upstream from said zone. The same considerations may apply to the control analyte.

The present invention may comprise an incubation zone in order to retain the liquid sample for a period of time, or serve to slow the flow of liquid prior to contact with the labelled binding reagents. The incubation zone serves to incubate the liquid sample with the one or more reagents thus optimising the pre-treatment and therefore the available amount of detectable analyte of interest. The incubation zone may for example comprise a flow barrier such as a breakable seal in order to retain the liquid sample and prevent it from flowing downstream. After a suitable period of time has elapsed, the flow barrier may be opened to allow liquid sample to flow downstream. A suitable period of time may range from 5 seconds to 1 hour. Alternatively the incubation zone may comprise a section of flow-path that is sufficiently long or slow-flowing such that the liquid sample is able to incubate for a period of time whilst flowing along the flow-path.

The assay device may comprise a liquid chamber for containing a liquid sample provided upstream from the labelled binding reagents. The liquid chamber may be provided upstream from a porous carrier or a microfluidic channel. Thus liquid applied to the liquid chamber may pass downstream to the porous carrier or microfluidic channel via an exit port. The liquid chamber may comprise the one or more reagents and/or the control analyte. Where more than one reagents is provided, they may be provided as a mixture, spaced apart from each another or next to each other. The one or more reagents may be provided on or contained within one or more porous or non-porous substrates. The substrate may be soluble in the liquid sample. The substrates may be spaced apart or provided, for example, in a stacked arrangement. Alternatively, the one or more reagents may be provided as lyophilised pellets or as a coating on an inner surface of the liquid chamber. The liquid chamber may include a liquid barrier which is typically provided at the point of exit of liquid from the liquid chamber. In the case where a liquid barrier is provided, the liquid chamber may further comprise a vent in order to allow the passage of liquid sample applied to the liquid chamber to the liquid barrier.

The control analyte may be provided on the same substrate as the one or more reagents or on a separate substrate. The substrates may be arranged one downstream from the other, such that liquid sample applied to the liquid chamber contacts a porous substrate, whereupon the liquid sample interacts with one or more reagents and with the control analyte.

A buffer zone may be provided downstream from the one or more reagents and upstream from the labelled binding reagents, for example in order to bring the pH of the treated liquid into the optimal working range of the assay. The buffer zone may be provided on a separate porous carrier material to and upstream from the first and second porous carrier materials.

In one embodiment, the assay device comprises a liquid chamber comprising the one or more reagents and the control analyte provided upstream from a porous carrier comprising the labelled binding reagents and detection and control zones. In an alternative embodiment, the porous carrier may comprise the reagents and the control analyte. The porous carrier may also comprise the buffer zone and the incubation zone.

Where the assay is provided as a kit, the control analyte and one or more reagents may be provided separately from the assay device. They may for example be provided in the dried state in a vial to which the liquid sample suspected of containing the analyte of interest may be added. After a suitable period, after which the liquid may be treated with a buffer if necessary, the treated liquid may then be added to the assay device. The labelled binding reagents may be included as part of the assay device or they may be added to the treated liquid following optional treatment by the buffer. Alternatively, the labelled binding reagents and buffer may be included as part of the assay device.

The species of interest may be any that requires a pre-treatment step in order to provide the analyte of interest in a detectable or more detectable form. The species may be chosen from a micro-organism including a bacterium, a virus or a fungus, including a yeast. The species may be harmful, i.e. pathogenic, or beneficial, i.e. commensal. The species may be for example a virus selected from Rhinovirus, Para influenza virus, Influenza type A, B or C virus, Respiratory syncytial virus (RSV), Coronavirus, Adenovirus, Coxsackie A virus, Herpes simplex virus, Enterovirus, Epstein-Barr virus, Cytomegalovirus, or Papillomavirus. It may be a bacteria, for example selected from Streptococci Group A, B, F or G, Enterococci, Chlamydia trachomatis, Campylobacter, E. Coli, salmonella, clostridium difficile, Clostridium botulinum, Staphylococcus aureus, Vibrio cholerae, Streptococci (X 2), Escherichia, and Porphyromonas.

The control species may be advantageously chosen from a species similar to that of the species of interest or from a species which requires a similar pre-treatment step in order to provide or enhance the provision of the analyte of interest. For example, if the species of interest is influenza A, the control species may be influenza B. If the species of interest is Streptococcus A, the control species may be Streptococcus B.

The analyte of interest may for example be a carbohydrate, a nucleic acid, a protein, or a lipid. The control analyte may be an antigen.

The liquid sample can be derived from any source, such as an industrial, environmental, agricultural, or biological source. The sample may be derived from or consist of a physiological source including blood, serum, plasma, interstitial liquid, saliva, sputum, ocular lens liquid, sweat, urine, milk, ascots liquid, mucous, synovial liquid, peritoneal liquid, transdermalexudates, pharyngeal exudates, bronchoalveolar lavage, tracheal aspirations, cerebrospinal liquid, semen, cervical mucus, vaginal or urethral secretions and amniotic liquid. In particular the analyte may be saliva, which may be in a diluted form. The liquid sample may be derived from a semi-solid or solid source by dilution, treatment or extraction into an aqueous liquid.

The flow-path may be a lateral flow porous carrier. Suitable materials that may be employed as a porous carrier include nitrocellulose, acetate fibre, cellulose or cellulose derivatives, polyester, polyolefin or glass fibre. The porous carrier may comprise nitrocellulose. This has the advantage that a binding reagent can be immobilised firmly without prior chemical treatment. If the porous solid phase material comprises paper, for example, the immobilisation of a binding reagent may be performed by chemical coupling using, for example, CNBr, carbonyldiimidazole, or tresyl chloride. A porous carrier material of choice on or in which the labelled binding reagents are provided is glass fibre. A porous carrier material of choice for providing a detection and control zone is nitrocellulose.

According to an exemplary embodiment, the porous carrier comprises three sections of partially overlapping porous carrier material. The first upstream section comprises a dried buffer in order to bring the pH of the treated liquid sample to an optimal level for the assay. The second section of porous carrier material comprises the labelled binding reagents for the analyte of interest and for the control analyte. The third section of porous carrier material comprises the detection and control zones. The first section of porous carrier may also serve to receive the liquid sample. Alternatively, a further section of porous carrier material may be provided upstream from the first section to receive the liquid sample.

The term binding reagent refers to a member of a binding pair, i.e., two different molecules wherein one of the molecules specifically binds with the second molecule through chemical or physical means. The two molecules are related in the sense that their binding with each other is such that they are capable of distinguishing their binding partner from other assay constituents having similar characteristics. The members of the specific binding pair are referred to as ligand and receptor (antiligand), a binding pair member and binding pair partner, and the like. A molecule may also be a binding pair member for an aggregation of molecules; for example, an antibody raised against an immune complex of a second antibody and its corresponding antigen may be considered to be an binding pair member for the immune complex.

In addition to antigen and antibody binding pair members, other binding pairs include, as examples without limitation, biotin and avidin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), and the like. Furthermore, specific binding pairs can include members that are analogues of the original specific binding member.

“Label” when used in the context of a labelled binding reagent, refers to any substance which is capable of producing a signal that is detectable by visual or instrumental means. Various labels useful in the present invention include labels which produce signals through either chemical or physical means, such as being optically detectable. Such labels include enzymes and substrates, chromogens, catalysts, fluorescent compounds, chemiluminescent compounds, electroactive species, dye molecules, radioactive labels and particle labels. The analyte itself may be inherently capable of producing a detectable signal. The label may be covalently attached to the binding reagent. In particular, the label may be chosen from one that is optically detectable.

The label may comprise a particle such as gold, silver, colloidal non-metallic particles such as selenium or tellurium, dyed or coloured particles such as a polymer particle incorporating a dye, or a dye sol. The dye may be of any suitable colour, for example blue. The dye may be fluorescent. Dye sols may be prepared from commercially-available hydrophobic dyestuffs such as Foron Blue SRP (Sandoz) and Resolin Blue BBLS (Bayer). Suitable polymer labels may be chosen from a range of synthetic polymers, such as polystyrene, polyvinyltoluene, polystyrene-acrylic acid and polyacrolein. The monomers used are normally water-insoluble, and are emulsified in aqueous surfactant so that monomer micelles are formed, which are then induced to polymerise by the addition of initiator to the emulsion. Substantially spherical polymer particles are produced. An ideal size range for such polymer particles is from about 0.05 to about 0.5 um. According to an exemplary embodiment, the label is a blue polymeric particle or a gold particle.

The one or more reagents may comprise nitrous acid generating reagents, for example, an acid and a nitrite (such as an alkali metal nitrite), which when combined in the presence of an aqueous sample form nitrous acid. The nitrous acid generating reagents may be provided on separate porous carriers. Such an embodiment is suitable for the detection of Streptococcae. The acid may be polymeric such as a polycarboxylic acid, a polysulphonic acid, a polystyrenesulphonic acid, a polyphosphoric acid, a polyacrylic acid, and a polymethacrylic acid. The acid may be chosen from for example citric acid, malonic acid, phenylacetic acid, oxalic acid, glycolic acid, chloroacetic acid, trichloroacetic acid, fluoroacetic acid, bomoacetic acid, iodaacetic acid, succinic acid, glutaric acid, adipic acid, pimelic acid, suberic acid, benzoic acid, benzene sulfonic acid, p-toluene sulphonic acid, azelaic acid and sebacic acid. Any suitable acid-generating reagent which is stable in the dry state and capable of reacting with the acid to produce nitrous acid may be used, such as an alkali metal nitrite.

The acid-generating reagent may include inorganic nitrites such as sodium, potassium, lithium, calcium, strontium, barium and silver nitrites, as well as organic nitrites such as butyl and isoamyl nitrites. In an exemplary embodiment, the acid-generating reagent comprises sodium nitrite. Additionally or alternatively, the reagent may comprise an enzyme such as Pronase B.

An assay device of the present invention may be used for the determination of the presence and/or amount of a Streptococcus species in a sample. The invention also provides an assay device for the determination of a Group A Streptococcus species comprising a Group B Streptococcus control species.

The present invention will now be described in more detail with reference to accompanying drawing, which illustrates an assay device in accordance with the invention.

An assay device (10) comprising an extraction chamber (18) provided upstream from a porous carrier test-strip (20). Dried reagents are provided within the chamber on porous discs (11, 12 and 13) which are stacked one upon the other in a vertical arrangement and held in position by a lip (17). In this particular embodiment, three discs are provided, although any number of discs may be provided as appropriate. Each disc typically comprises different reagents. Liquid sample applied to the sample extraction chamber passes through the porous discs at the same time interacting with the reagents present. Treated liquid sample thereafter passes to the porous carrier via the lower tapered section (15) of the extraction chamber. There may also be a liquid sample incubation means (not shown), such as a breakable seal or a time gate, present within the device in order to incubate treated liquid sample for a period of time before it passes to the porous carrier. The liquid sample incubation means would typically be provided at the exit region (19) of the extraction chamber (18). Also shown is a vent (14), which may be optional, to allow liquid sample to pass into the lower part of the chamber in the case where the exit region is sealed. The porous carrier test strip (20) comprises three sections which overlap each other at regions (22) and (23). The first upstream section comprises a buffer pad, the second section comprises the labelled binding reagent and the third downstream section comprises the detection zone (25) and the control zone (26).

### EXAMPLE

### Preparation of an Assay Kit Comprising a Control Means

A sample liquid extraction chamber was provided comprising a plastic hollow cylinder 33 mm in length and an inner diameter of 9 mm tapering to 4 mm at one end. Three 9 mm diameter Porex discs (80-120μ pore-size) were prepared each comprising dried reagents. Disc 1 was prepared by application of 30 μL of 2M citric acid to the disc followed by drying. Disc 2 was prepared by application of 30 μL of 4M aq. sodium nitrite. Disc 3 was prepared by application of 30 μL of 4M aq. sodium nitrite with 8.33×10⁶ cfu/mL of heat-inactivated Group B Streptococcus cells followed by drying. The three discs were placed into the plastic hollow cylinder with disc 3 placed upstream from and on top of disc 2 which was placed upstream from and on top of disc 1.

A lateral flow test-strip was prepared by as follows:

A buffer conjugate pad porous carrier (5 mm width by 10 mm length) was prepared by application of 30 μL of 1.5M trizma base dried onto glass fibre (Millipore G041). A conjugate pad porous carrier (5 mm width by 10 mm length) was prepared by application of 30 μL of 1.0 OD anti-GBS gold conjugate and 30 μL 1.0 OD anti-GAS gold conjugate dried onto glass fibre (Millipore G041). A porous carrier was prepared by printing onto nitrocellulose (5 mm width by 40 mm length, Millipore HF-90 backed with 7 mil Mylar), 2.0 mg/ml anti-group A streptococcus at 1.0 μL/cm to form the detection zone and by printing 2.0 mg/ml anti-group B streptococcus at 1.0 μL/cm to form the control zone. The detection zone was positioned 10 mm along the length of the nitrocellulose porous carrier and the control zone positioned 16 mm along the length of the nitrocellulose porous carrier and downstream from the detection zone.

The buffer pad was partially overlaid on and upstream from the conjugate pad which was partially overlaid on and upstream from the nitrocellulose carrier. A sink pad (Whatman GB003) was provided downstream from the nitrocellulose carrier to form the completed test-strip.

The extraction chamber was placed with the tapered end lowermost into an Eppendorf tube. 400 μL of liquid sample comprising Strep A was added to the extraction chamber comprising the Porex discs and allowed to incubate for 5 minutes in the Eppendorf tube. Thereafter 80 μL of liquid extract from the Eppendorf tube was applied to the buffer pad of the test strip. The detection and control zones were read by eye after 5 minutes.

### RESULTS

A signal was detected at the control zone indicating that liquid sample had been successfully applied to the assay kit, the assay binding reagents were working satisfactorily and that the extraction reagents had worked satisfactorily. A signal was also detected at the detection zone indicating the presence of strep A in the liquid sample.

1. An assay device for determining the presence and/or amount of an analyte of interest in a liquid sample, said assay device comprising a liquid flow-path comprising:
(a) one or more reagents which, when contacted with the liquid sample, provide the analyte of interest in a detectable or more detectable form;
(b) a control substance, comprising a control analyte, which, when contacted with said one or more reagents provides the control analyte in a detectable or more detectable form;
(c) a detection zone for detecting a first labelled binding reagent for the analyte of interest, provided downstream from the one or more reagents; and
(d) a control zone for detecting a second labelled binding reagent for the control analyte, provided downstream from the control analyte and from the one or more reagents;
wherein detection of the first labelled binding reagent at the detection zone is indicative of the presence and/or amount of the analyte of interest and detection of the second labelled binding reagent at the control zone is indicative of the effectiveness of the one or more reagents in providing the analyte of interest in a detectable or more detectable form.

2. An assay device according to claim 1, wherein the detection zone comprises an immobilised binding reagent for the analyte of interest and/or the control zone comprises an immobilised binding reagent for the control analyte.

3. An assay device according to claim 1, wherein the flow-path comprises a porous carrier.

4. An assay device according to claim 3, wherein the porous carrier comprises the detection zone and the control zone.

5. An assay device according to claim 1, wherein the one or more reagents and/or the control analyte is/are provided on and/or in the porous carrier.

6. An assay device according to claim 1, comprising a liquid sample chamber provided upstream of the porous carrier.

7. The assay device according to claim 4 wherein the liquid sample chamber comprises said one or more reagents and/or the control substance.

8. An assay device according to claim 1, comprising the first labelled binding reagent provided upstream from the detection zone and downstream from the one or more reagents, and the second labelled binding reagent provided upstream from the control zone and downstream from the one or more reagents and the control substance.

9. An assay device according to claim 8, comprising a first porous carrier material comprising the first and second labelled binding reagents provided upstream from a second porous carrier material comprising the detection and control zones.

10. An assay device according to claim 1, comprising a buffer zone provided downstream from the one or more reagents and upstream from the first and second labelled binding reagents.

11. An assay device according to claim 1, wherein the buffer zone is provided on a separate porous carrier material to and upstream from the first and second porous carrier materials.

12. An assay device according to claim 1, wherein the one or more reagents serve to extract the analyte the interest.

13. An assay device according claim 12, wherein the one or more reagents comprise nitrous acid generating reagents.

14. An assay device according to claim 13, wherein the nitrous acid generating reagents comprise an alkali metal nitrite and an acid.

15. An assay device according to claim 1, wherein the nitrous acid generating reagents are provided on separate porous carriers.

16. An assay device according to claim 1 for the determination of the presence and/or amount of a Streptococcus species in a sample.

17. An assay device for the determination of a Group A Streptococcus species comprising a Group B Streptococcus control species.

18. A method for the determination of the presence and/or amount of an analyte of interest in a liquid sample, comprising:
(a) applying the sample to an assay device comprising one or more reagents and a control substance which comprises or consists of a control analyte, wherein the one or more reagents interacts with the liquid sample and with the control substance to provide the analyte of interest and the control analyte in a detectable or more detectable form;
(b) causing the analyte of interest to interact with a first labelled binding reagent and the control analyte to interact with a second labelled binding reagent;
(c) detecting the first labelled binding reagent at a detection zone and the second labelled binding reagent at a control zone;
wherein detection of the first labelled binding reagent at the detection zone is indicative of the presence or amount of the analyte of interest and wherein detection of the second labelled binding reagent at the control zone is indicative of the effectiveness of the one or more reagents in providing the analyte of interest in a detectable or more detectable form.
